(19) United States
(12) Patent Application Publication
(10) Pub. No.: US 2023/0241041 A1
(43) Pub. Date: Aug. 3, 2023

(54) LURBINECTEDIN IN THE TREATMENT OF MALIGNANT MESOTHELIOMA

(71) Applicant: PHARMA MAR, S.A., Madrid (ES)

(72) Inventors: Ioannis Metaxas, Bern (CH); Roger Von Moos, Bern (CH)

(21) Appl. No.: 17/640,315

(22) PCT Filed: Sep. 3, 2020
(86) PCT No.: PCT/EP2020/074689

§ 371 Date: Mar. 3, 2022

(30) Foreign Application Priority Data
Sep. 3, 2019 (EP) 19382749.0

(51) Int. Cl. A61K 31/4365; A61P 35/00; A61K 38/19; A61K 45/06

(52) U.S. Cl. CPC A61K 31/4365; A61K 38/193; A61K 45/06; A61P 35/00

(57) ABSTRACT

The use of Lurbinectedin in the treatment of malignant mesothelioma is provided.

### FIELD OF THE INVENTION

The present invention relates to the treatment of cancer and, in particular to the treatment of malignant mesothelioma by using lurbinectedin.

### BACKGROUND OF THE INVENTION

Malignant mesothelioma is a disease in which malignant (cancer) cells are found in the pleura (the thin layer of tissue that lines the chest cavity and covers the lungs) or the peritoneum (the thin layer of tissue that lines the abdomen and covers most of the organs in the abdomen). Malignant pleural mesothelioma (MPM) is relatively rare but aggressive. MPM is closely related to asbestos exposure, a relationship first described in the early ‘60s. Because the latent period can be between 30 and 50 years, and because asbestos had been used in Western countries until recently in a plethora of applications, such as home insulation, an increased incidence of mesothelioma is expected to peak in the coming decade. Symptoms of MPM include breathlessness, cough, chest pain, fatigue, fevers and weight loss. Once it develops, MPM is usually associated with a poor rate of survival. However, the length of survival depends on factors including the extent of the tumor at diagnosis (“tumor stage”), the type of mesothelioma and the patient’s response to treatment. Although mesothelioma is usually not curable, the length of survival can range from a few months to a couple of years. Treatment of this cancer depends on how far the cancer has spread once it has been diagnosed. The aim of treatment may be “curative”, i.e., to remove all of the disease, or “palliative,” i.e., to alleviate symptoms. The aim of treatment may be to reduce the cancer burden and/or to delay disease progression.

Platinum-pemetrexed chemotherapy with or without surgery, and potentially additional radiotherapy, is considered as standard first-line therapy for MPM. Still, even such a tri-modal treatment provides a progression-free survival (PFS) of less than 12 months, which actually renders MPM an incurable disease for the majority of the patients.

On the other hand, a large proportion of progressive patients remain in good health for further systemic treatment. The most commonly used agents are navelbine or gemcitabine, but this decision is hardly evidence-based as older and modern-era trials have repeatedly shown modest activity for both compounds with a response rate of less than 10%, median PFS of no more than 2-3 months and overall survival (OS) of less than 9 months.

Many clinical trials with novel agents have been conducted to improve outcome of progressive MPM. The vast majority of them failed to provide a meaningful, “standard” second-line treatment, as results were again disappointing (response constitutively <10%, PFS <3 months and OS 10 months at best), as reported by Buikhuisen et al. (Lung Cancer, 2015, 89, 223-231). Only pemetrexed-naive patients receiving pemetrexed upon progression had an interesting outcome, but after this molecule had become standard first-line treatment, its role as re-challenge became more obscure and retrospective trials now point for using it mostly in cases with a clearly long PFS on prior pemetrexed.

With the advent of immunotherapy several trials examine the role of anti-PD-1, anti-PD-L1 and anti-CTLA-4 antibodies. Although data from early Phase I/II trials are promising, as disclosed by Scherpereel et al. (Lancet Oncol in press, 2018) and Disselhorst et al. (Lancet Respir Med in press, 2018), other immunotherapy studies and retrospective analyses challenge its role as reported by Metaxas (J. Thor. Oncol. 2018 Nov; 13(11): 1784-1791) and Maio (Lancet Oncol, 2017, 18, 1261-1273).

In any case, all these data underline the high-unmet medical need for new approaches in malignant mesothelioma, particularly MPM, more particularly progressive MPM. Therefore, the development of novel therapies and agents to treat patients suffering from this disease is urgently needed.

Lurbinectedin, also known as PM01183 and initially called tryptamicidin, is a synthetic antitumoral compound, and the subject of WO 03/014127. The chemical structure of lurbinectedin is represented as follows:

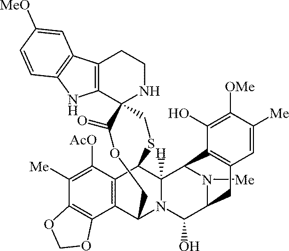 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

Lurbinectedin has demonstrated highly potent in vitro activity against solid and non-solid tumour cell lines as well as significant in vivo activity in several xenografted human tumor cell lines in mice, such as those for breast, kidney and ovarian cancer. It is a selective inhibitor of the oncogenic transcription programs on which many tumors are particularly dependent. Together with its effect on cancer cells, lurbinectedin inhibits oncogenic transcription in tumor-associated macrophages, downregulating the production of cytokines that are essential for the growth of the tumor. Transcriptional addiction is an acknowledged target in those diseases, many of them lacking other actionable targets.

### SUMMARY OF THE INVENTION

In a first aspect of the present invention there is provided lurbinectedin or a pharmaceutically acceptable salt or ester thereof for use in the treatment of malignant mesothelioma, wherein lurbinectedin is administered as a monotherapy.

It has surprisingly been discovered that lurbinectedin or a pharmaceutically acceptable salt or ester thereof as a monotherapy is effective in the treatment of malignant mesothelioma, with data showing sensitivity to this compound. Thus, the need for a new and effective treatment of malignant mesothelioma is met by the present invention.

In a further aspect of the present invention there is provided the use of lurbinectedin or a pharmaceutically acceptable salt or ester thereof in the manufacture of a medicament for the treatment of malignant mesothelioma, wherein lurbinectedin is administered as a monotherapy.

In a further aspect of the present invention there is provided a method of treating malignant mesothelioma, in a patient in need thereof comprising administering to said patient an effective amount of lurbinectedin or a pharmaceutically acceptable salt or ester thereof as a monotherapy.

In a further aspect of the present invention there is provided lurbinectedin or a pharmaceutically acceptable salt or ester thereof for use in the treatment of malignant mesothelioma; wherein lurbinectedin treatment excludes treatment with a combination of lurbinectedin and a platinum agent.

In a further aspect of the present invention there is provided lurbinectedin or a pharmaceutically acceptable salt or ester thereof for use in the treatment of malignant mesothelioma, wherein lurbinectedin treatment excludes a combination of lurbinectedin and cisplatin.

In a further aspect of the present invention there is provided lurbinectedin or a pharmaceutically acceptable salt or ester thereof as sole chemotherapy agent for use in the treatment of malignant mesothelioma.

In a further aspect of the present invention there is provided lurbinectedin or a pharmaceutically acceptable salt or ester thereof for use in the treatment of malignant mesothelioma; wherein lurbinectedin is the sole chemotherapy agent; and wherein lurbinectedin is administered following immunotherapy; preferably an anti-PD-1, anti-PD-L1 or anti CTLA-4 therapy, or combinations thereof.

In a further aspect of the present invention there is provided a kit comprising lurbinectedin or a pharmaceutically acceptable salt or ester thereof together with instructions for treating malignant mesothelioma.

The following features apply to all aspects of the invention.

The malignant mesothelioma may be malignant pleural mesothelioma. The malignant mesothelioma may be malignant peritoneal mesothelioma. The malignant mesothelioma may be pericardial mesothelioma. The malignant mesothelioma may be malignant testicular mesothelioma. Preferably, the malignant mesothelioma is malignant pleural mesothelioma.

The malignant mesothelioma may be progressive. In a preferred embodiment, the malignant mesothelioma is progressive.

The malignant mesothelioma may have progressed from first-line therapy, preferably from standard first-line therapy. Standard therapy may be platinum-pemetrexed chemotherapy. Standard therapy may also include surgery. Standard therapy may also include radiotherapy.

The malignant mesothelioma may have progressed from immunotherapy. The immunotherapy may be an anti-PD-1, anti-PD-L1 or anti CTLA-4 therapy, or a combination thereof.

The use of lurbinectedin according to the present invention may be as a second-line therapy. The use of lurbinectedin according to the present invention may be as a third-line therapy, including wherein the second-line therapy is immunotherapy.

The use of lurbinectedin according to the present invention may be as a first-line therapy.

The lurbinectedin according to the present invention may be administered in cycles once every one to four weeks, preferably once every three weeks.

The lurbinectedin according to the present invention may be administered at a dose of 1 to 5 mg/m² body surface area, 2 to 3 mg/m² body surface area, about 3 mg/m² body surface area, 3 to 3.5 mg/m² body surface area, or 3.2 mg/m² body surface area.

The lurbinectedin according to the present invention may be administered as an infusion, preferably with an infusion time of up to 24 hours, 1 to 12 hours, 1 to 6 hours and most preferably 1 hour.

The patient may additionally be treated with radiotherapy.

The radiotherapy may be administered prior to or subsequent to administration of lurbinectedin or a pharmaceutically acceptable salt or ester thereof, preferably at least an hour, three hours, five hours, 12 hours, a day, a week, a month, more preferably several months (e.g. up to three months) prior or subsequent to administration of lurbinectedin or a pharmaceutically acceptable salt or ester thereof.

The patient may additionally be treated with an anti-emetic, G-CSF and/or GM-CSF. The anti-emetic, G-CSF and/or GM-CSF may be independently administered prior to or subsequent to administration of lurbinectedin or a pharmaceutically acceptable salt or ester thereof.

The malignant mesothelioma may be epithelioid mesothelioma.

The malignant mesothelioma may be sarcomatoid mesothelioma.

The malignant mesothelioma may be biphasic mesothelioma.

The lurbinectedin according to the present invention may be administered in the form of a pharmaceutically acceptable salt selected from the hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate p-toluenesulfonate, sodium, potassium, calcium and ammonium salts, ethylenediamine, ethanolamine, N,N-dialkylenethanolamine, triethanolamine and basic aminoacids salts.

The lurbinectedin may be comprised in a pharmaceutical composition which also comprises a disaccharide. The pharmaceutical composition may be a lyophilized pharmaceutical composition.

The present invention has therefore surprisingly established a new and effective treatment for malignant mesothelioma, particularly malignant pleural mesothelioma. The present invention has especially established a new and effective treatment for the challenging patients who have experienced disease progression, particularly disease progression after standard treatment, most particularly disease progression after standard treatment based on platinum-pemetrexed chemotherapy.

### BRIEF DESCRIPTION OF THE FIGURES

FIG. 1 is a Kaplan-Meier curve of progression-free survival (PFS) in all patients.

FIG. 2 is a Kaplan-Meier curve of overall survival (OS) in all patients.

FIG. 3 is a Kaplan-Meier curve comparing PFS in patients with epithelioid and non-epithelioid MPM.

FIG. 4 is a Kaplan-Meier curve comparing OS in patients with epithelioid and non-epithelioid MPM.

FIG. 5 is a Kaplan-Meier curve comparing PFS in patients prior-treated with immunology and those not prior-treated with immunology.

FIG. 6 is a Kaplan-Meier curve comparing OS in patients prior-treated with immunology and those not prior-treated with immunology.

FIG. 7 is a Kaplan-Meier curve comparing PFS in patients with a < 6 months and ≥ 6 months progression-free interval on prior platinum-pemetrexed chemotherapy.

FIG. 8 is a Kaplan-Meier curve comparing OS in patients with a < 6 months and ≥ 6 months progression-free interval on prior platinum-pemetrexed chemotherapy.

### EMBODIMENTS OF THE INVENTION

In the present application, a number of general terms and phrases are used, which should be interpreted as follows.

The term “treating”, as used herein, unless otherwise indicated, means reversing, attenuating, alleviating or inhibiting the progress of the disease or condition to which such term applies, or one or more symptoms of such disorder or condition. The term “treatment”, as used herein, unless otherwise indicated, refers to the act of treating as “treating” is defined immediately above.

“Patient” includes humans, non-human mammals (e.g., dogs, cats, rabbits, cattle, horses, sheep, goats, swine, deer, and the like) and non-mammals (e.g., birds, and the like). In some embodiments, the patient is a human.

Lurbinectedin is a synthetic alkaloid, having the following structure:

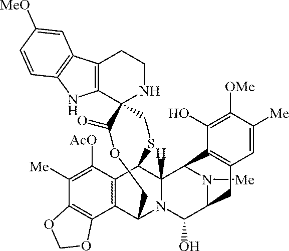 [SMILES: COc1ccc2[nH]c3c(c2c1)CCN[C@]31CS[C@@H]2c3c(OC(C)=O)c(C)c4c(c3[C@H](COC1=O)N1[C@H]2[C@H]2c3c(cc(C)c(OC)c3O)C[C@@H]([C@@H]1O)N2C)OCO4]

Lurbinectedin has been already tested in clinical trials with promising results in different entities and is now evaluated in a Phase III study in relapsed small cell lung cancer.

Information regarding its mechanism of action and in vivo efficacy can be found in 100^{th} AACR Annual Meeting, April 18-22, 2009, Denver, CO, Abstract Nr. 2679 and Abstract Nr. 4525; Leal JFM et. al. Br. J. Pharmacol. 2010, 161, 1099-1110; and Belgiovine, C et al. Br. J. Cancer, 2017; 117(5): 628-638;

Further information regarding the clinical development of PM01183 can be found in:
Elez, ME. et. al. Clin. Cancer Res. 2014, 20(8), 2205-2214;
50^{th} ASCO Annual Meeting, May 30 - Jun. 3, 2014, Chicago, IL, Abstract 5505;
26^{th} EORTC-NCI-AACR Symposium on Molecular Targets and Cancer Therapeutics; November 18-21, 2014, Barcelona, Spain, published in Eur. J. Cancer 2014, 50 (Suppl. 6), pages 13-14, Abs. No. 23.
51^{th} ASCO Annual Meeting, May 29 - Jun. 2, 2015, Chicago, IL, Abstract No. TPS2604 and Abstract Nr. 7509, published in J. Clin. Oncol. 33, 2015 (suppl);
54^{th} ASCO Annual Meeting, June 1-5, 2018, Chicago, IL, Abstract No. 11519, published in J. Clin. Oncol. 36, 2018 (suppl);
Cruz, C. et al. J. Clin. Oncol. 2018; in press 1-21;
54^{th} ASCO Annual Meeting, June 1-5, 2018, Chicago, IL, Abstract No. 8570, published in J. Clin. Oncol. 36, 2018 (suppl);

Lurbinectedin was assessed in a Phase I trial in combination with cisplatin in several tumor types. Metaxas et al. (Lung Cancer 2016, 12, 136-138) reports on preliminary toxicity and efficacy data for two patients with malignant mesothelioma within said Phase I trial. Metaxas concluded that “the combination of cisplatin with the novel compound lurbinectedin appears safe to administer in malignant mesotheliomas and shows promising efficacy as further systemic palliative treatment. For this reason, clinical trials using this combination for mesothelioma patients should be evaluated“. Metaxas provides no suggestion that lurbinectedin might be effective as a monotherapy.

The term “lurbinectedin” is intended here to cover any pharmaceutically acceptable salt, ester, solvate, hydrate, prodrug, or any other compound which, upon administration to the patient is capable of providing (directly or indirectly) the compound as described herein. However, it will be appreciated that non-pharmaceutically acceptable salts also fall within the scope of the invention since those may be useful in the preparation of pharmaceutically acceptable salts. The preparation of salts can be carried out by methods known in the art.

For instance, pharmaceutically acceptable salts of the compounds provided herein are synthesized from the parent compounds, which contain a basic or acidic moiety, by conventional chemical methods. Generally, such salts are, for example, prepared by reacting the free acid or base of these compounds with a stoichiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

Any compound that is a prodrug of Lurbinectedin is within the scope and spirit of the invention. The term “prodrug” is used in its broadest sense and encompasses those derivatives that are converted in vivo to PM01183. The prodrug can hydrolyze, oxidize, or otherwise react under biological conditions to provide PM01183. Examples of prodrugs include, but are not limited to, derivatives and metabolites of PM01183 that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by Burger in “Medicinal Chemistry and Drug Discovery” 6th ed. (Donald J. Abraham ed., 2001, Wiley) and “Design and Applications of Prodrugs” (H. Bundgaard ed., 1985, Harwood Academic Publishers).

In addition, any drug referred to herein may be in crystalline or amorphous form either as free compounds or as solvates (e.g. hydrates) and it is intended that all forms are within the scope of the present invention. Methods of solvation are generally known within the art.

Moreover, Lurbinectedin for use in accordance with the present invention may be prepared following the synthetic process such as the one disclosed in WO 03/014127, which is incorporated herein by reference.

“Malignant mesothelioma” is a disease in which malignant (cancer) cells are found in the pleura (the thin layer of tissue that lines the chest cavity and covers the lungs) or the peritoneum (the thin layer of tissue that lines the abdomen and covers most of the organs in the abdomen). Malignant mesothelioma may also form in the heart or testicles, but this is rare. The four types of mesothelioma are therefore pleural (lung lining), peritoneal (abdominal lining), pericardial (heart sac) and testicular.

Mesothelioma can also be identified by three cancer cell types: epithelioid, sarcomatoid and biphasic, and can therefore be defined as epithelioid mesothelioma (epithelioid cells), sarcomatoid mesothelioma (sarcomatoid cells) or biphasic mesothelioma (epithelioid and sarcomatoid cells).

Pleural is the most common mesothelioma. Approximately 70% to 75% of cases occur in the pleura. Peritoneal disease accounts for 10% to 20% of mesothelioma cases. There is less research available on peritoneal compared to pleural; however, the prognosis for this tumor type is better. Pericardial Mesothelioma is extremely rare. Around 200 cases are reported in medical literature. Testicular mesothelioma develops in the lining of the testes. This form of mesothelioma is the most rare. Less than 100 cases are reported in the medical literature.

The three mesothelioma cell varieties are epithelial, sarcomatoid and biphasic. Biphasic is a mix of the first two cell types. Different mesothelioma tumors respond differently to treatment. Epithelial or epithelioid cells typically respond the best to treatment, and sarcomatoid cells are typically more resistant to treatment.

Epithelioid mesothelioma makes up approximately 70% to 75% of all cases of asbestos-related mesothelioma cancers. Epithelioid cell typically has the best prognosis. It tends to be less aggressive and doesn’t spread as quickly as sarcomatoid and biphasic cell disease. About 50% of pleural disease is epithelioid. Around 75% of peritoneal tumors are made up of epithelioid cells.

Sarcomatoid is the least common mesothelioma cell category. It is typically the most aggressive and difficult to treat. It accounts for around 10% to 20% of all mesothelioma diagnoses. About 20% of pleural tumors are sarcomatoid, while only 1% of peritoneal mesothelioma are sarcomatous.

Biphasic mesothelioma refers to tumors that contain epithelial and sarcomatoid cells. Life expectancy after diagnosis with biphasic mesothelioma depends upon which cell predominates in the tumor. More epithelioid cells generally mean a better prognosis. If the tumor is mostly sarcomatous, it is harder to treat and life expectancy is shorter. Around 30% of pleural and 25% of peritoneal tumors are biphasic cell.

Prevalence of Mesothelioma Tumors by Cell Type

| Cell Type | Pleural | Peritoneal |
|---|---|---|
| Epithelioid | 50% | 75% |
| Biphasic | 30% | 24% |
| Sarcomatoid | 20% | 1% |

Based on the limited number of cases reported in the medical literature, pericardial mesothelioma exhibits roughly equal distribution of the three mesothelioma cell types. Approximately two-thirds of testicular mesothelioma cases are epithelioid cell. The rest of testicular cases are biphasic. Only one case of purely sarcomatoid cell disease is reported for testicular mesothelioma.

The present invention is preferably the use of lurbinectedin for the treatment of malignant pleural mesothelioma (MPM).

The malignant mesothelioma to be treated may be epithelioid. The malignant mesothelioma to be treated may be sarcomatoid. The malignant mesothelioma to be treated may be biphasic. The present invention has identified that treatment with lurbinectedin according to the present invention equalises the prognosis of sarcomatoid and epithelioid malignant mesothelioma. Given the worse prognosis for sarcomatoid (including biphasic) cancers, the present invention therefore has particular benefits for patients having sarcomatoid or biphasic malignant mesothelioma.

“Progressive malignant mesothelioma” is where the disease has progressed after first-line therapy. In an embodiment, the present invention is directed to treatment of patients who experience progression after standard treatment.

“First-line therapy” means the initial treatment given to the patient. Standard first line therapy of malignant mesothelioma is typically platinum-pemetrexed chemotherapy with or without surgery, and potentially additional radiotherapy. Thus, standard first-line therapy may comprise platinum-pemetrexed chemotherapy, platinum-pemetrexed chemotherapy and surgery, platinum-pemetrexed chemotherapy and radiotherapy or platinum-pemetrexed chemotherapy and surgery plus radiotherapy.

The first-line therapy may alternatively be an immunotherapy or platinum-pemetrexed chemotherapy and an immunotherapy. Immunotherapy may be an anti-PD-1, anti-PD-L1 or anti-CTLA-4 therapy, or combinations thereof, for example antibody therapeutics.

The first-line therapy may alternatively be a different therapy, for example an anti-angiogenesis therapy, including a VEGF inhibitor such as bevacizumab.

Progressive therapy according to the present invention may therefore be after platinum-pemetrexed chemotherapy with or without surgery, and potentially additional radiotherapy. Progressive therapy according to the present invention may be after: platinum-pemetrexed chemotherapy and immunotherapy; immunotherapy; other therapies, for example after treatment with an anti-angiogenesis therapy, including a VEGF inhibitor such as bevacizumab.

Progressive treatment may be a second line therapy. Progressive treatment may be a third or further line treatment, for example following a number of the therapies outlined above.

For example, where progressive therapy according to the present invention is a third-line therapy, the second line therapy may be an immunotherapy.

“Monotherapy” means the patient is treated with lurbinectedin as the sole chemotherapeutic agent and not in combination. For example, the patient is treated with lurbinectedin alone and not lurbinectedin in combination with a platinum agent, for example cisplatin. Lurbinectedin monotherapy does not, however, preclude the patient from other medicaments such as, for example, an anti-emetic. In embodiments, lurbinectedin monotherapy may include radiotherapy.

The anti-emetic may be a corticosteroid or serotonin (5-HT3) antagonists. In embodiments, other medicaments include granulocyte colony-stimulating factor (G-CSF) or granulocyte macrophage colony-stimulating factor (GM-CSF). Thus, in some embodiments, lurbinectedin monotherapy includes administration of an anti-emetic, G-CSF or GM-CSF. In some embodiments, lurbinectedin monotherapy includes administration of an anti-emetic and/or G-CSF. In some embodiments, lurbinectedin monotherapy includes administration of G-CSF. The administration of G-CSF has been shown to diminish the incidence of grade 3-4 neutropenia.

As discussed above, the patient can be treated with lurbinectedin and other treatments. In an embodiment of the present invention, there is provided lurbinectedin or a pharmaceutically acceptable salt or ester thereof for use in the treatment of malignant mesothelioma, wherein treatment with lurbinectedin excludes combination treatment of lurbinectedin and a platinum agent. In an embodiment of the present invention, there is provided lurbinectedin or a pharmaceutically acceptable salt or ester thereof for use in the treatment of malignant mesothelioma, wherein treatment with lurbinectedin excludes combination treatment of lurbinectedin and cisplatin. Thus, these embodiments do not exclude platinum therapy (for example, cisplatin therapy) being used in prior lines of therapy (for example, first-line therapy).

“Radiotherapy” means that in a further embodiment of the present invention, the patient in need of said treatment is given radiation therapy with (including prior to, during or after) treatment with lurbinectedin. In embodiments of the present invention, the patient is treated with lurbinectedin or a pharmaceutically acceptable salt or ester thereof and radiotherapy. In an embodiment, the radiation therapy is administered prior or subsequent to administration of lurbinectedin or a pharmaceutically acceptable salt or ester thereof, preferably at least an hour, three hours, five hours, 12 hours, a day, a week, a month, more preferably several months (e.g. up to three months) prior or subsequent to administration of lurbinectedin or a pharmaceutically acceptable salt or ester thereof.

Lurbinectedin may be administered to the patient at a dose of 1 to 5 mg/m² body surface area, 2 to 3 mg/m² body surface area, about 3 mg/m² body surface area, 3 to 3.5 mg/m² body surface area, or 3.2 mg/m² body surface area.

In embodiments of the invention, in the case of grade ≥2 toxicity, treatment is withheld until resolution to grade 0-1. For grade 3-4 toxicity, the dose may be reduced to 2.6 mg/m2 q3w (first occurrence) or 2.0 mg/m2 (second occurrence). In the case of a third re-emergence of grade 3-4 toxicity, treatment may be permanently withheld.

In relation to leukopenia grade 3-4 and/or febrile neutropenia, secondary granulocyte colony-stimulating factor (G-CSF) prophylaxis may be instituted. This may be accompanied with a dose reduction by one level.

Pharmaceutical compositions comprising lurbinectedin or a pharmaceutically acceptable salt or ester thereof, and a pharmaceutically acceptable carrier may be formulated according to the chosen route of administration. Examples of the administration form include without limitation oral, topical, parenteral, sublingual, rectal, vaginal, ocular and intranasal. Parenteral administration includes subcutaneous injections, intravenous, intramuscular, intrasternal injection or infusion techniques. Preferably the compositions are administered parenterally. Pharmaceutical compositions of the invention can be formulated so as to allow a compound according to the present invention to be bioavailable upon administration of the composition to an animal, preferably human. Compositions can take the form of one or more dosage units, where for example, a tablet can be a single dosage unit, and a container of a compound according to the present invention may contain the compound in liquid or in aerosol form and may hold a single or a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example inhalatory administration. Powders may also be used for inhalation dosage forms. The term “carrier” refers to a diluent, adjuvant or excipient, with which the compound according to the present invention is administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In one embodiment, when administered to an animal, the compounds and compositions according to the present invention, and pharmaceutically acceptable carriers are sterile. Water is a preferred carrier when the compounds according to the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more for the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agent such as sucrose or saccharin; a flavoring agent such as peppermint, methyl salicylate or orange flavoring; and a coloring agent.

When the composition is in the form of a capsule (e.g. a gelatin capsule), it can contain, in addition to materials of the above type, a liquid carrier such as polyethylene glycol, cyclodextrins or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intracerebral, intraventricular, intrathecal, intravaginal or transdermal. The preferred mode of administration is left to the discretion of the practitioner, and will depend in part upon the site of the medical condition. In a more preferred embodiment, the compounds according to the present invention are administered intravenously. Infusion times of up to 24 hours are preferred to be used, more preferably 1 to 12 hours, with 1 to 6 hours being most preferred. Short infusion times which allow treatment to be carried out without an overnight stay in a hospital are especially desirable. However, infusion may be 12 to 24 hours or even longer if required. Infusion may be carried out at suitable intervals of, for example, 1 to 4 weeks, preferably once every three weeks.

The liquid compositions of the invention, whether they are solutions, suspensions or other like form, can also include one or more of the following: sterile diluents such as water for injection, saline solution, preferably physiological saline, Ringer’s solution, isotonic sodium chloride, fixed oils such as synthetic mono or diglycerides, polyethylene glycols, glycerin, or other solvents; antibacterial agents such as benzyl alcohol or methyl paraben; and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant.

The compositions comprise an effective amount of a Lurbinectedin such that a suitable dosage will be obtained. The correct dosage of the Lurbinectedin will vary according to the particular formulation, the mode of application, and its particular site and host. Other factors like age, body weight, sex, diet, time of administration, rate of excretion, condition of the host, drug combinations, reaction sensitivities and severity of the disease should be taken into account. Administration can be carried out continuously or periodically within the maximum tolerated dose.

The dose will be selected according to the dosing schedule, having regard to the existing data on Dose Limiting Toxicity, on which see for example the above mentioned Phase I studies cited in the background of the invention. These documents are also incorporated herein in full by specific reference.

Typically, the amount is at least about 0.01% of lurbinectedin, and may comprise at least 80%, by weight of the composition. When intended for oral administration, this amount can be varied to range from about 0.1% to about 80% by weight of the composition. Preferred oral compositions can comprise from about 4% to about 50% of lurbinectedin by weight of the composition.

Preferred compositions of the present invention are prepared so that a parenteral dosage unit contains from about 0.01% to about 10% by weight of lurbinectedin. More preferred parenteral dosage unit contains about 0.5% to about 5% by weight of lurbinectedin.

For intravenous administration, the composition is suitable for doses from about 0.01 mg/kg to about 250 mg/kg of the animal’s body weight, preferably from about 0.01 mg/kg and about 20 mg/kg of the animal’s body weight, more preferably from about 0.01 mg/kg to about 10 mg/kg, about 0.01 mg/kg to about 1 mg/kg, about 0.01 mg/kg to about 0.5 mg/kg, about 0.01 mg/kg to about 0.2 mg/kg, about 0.05 mg/kg to about 0.2 mg/kg, about 0.08 mg/kg to about 0.2 mg/kg, about 0.07 mg/kg to about 0.15 mg/kg, about 0.07 mg/kg to about 0.12 mg/kg, about 0.07 mg/kg to about 0.1 mg/kg, about 0.08 mg/kg to about 0.09 mg/kg of the animal’s body weight.

Lurbinectedin can be administered by any convenient route, for example by infusion or bolus injection, by absorption through epithelial or mucocutaneous linings.

In specific embodiments, it can be desirable to administer lurbinectedin, or compositions locally to the area in need of treatment. In one embodiment, administration can be by direct injection at the site (or former site) of a cancer, tumor or neoplastic or pre-neoplastic tissue.

Pulmonary administration can also be employed, e.g. by use of an inhaler or nebulizer, and formulation with an aerosolizing agent, or via perfusion in a fluorocarbon or synthetic pulmonary surfactant. In certain embodiments, lurbinectedin can be formulated as a suppository, with traditional binders and carriers such as triglycerides.

The present compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington’s Pharmaceutical Sciences” by E. W. Martin.

The pharmaceutical compositions can be prepared using methodology well known in the pharmaceutical art. For example, a composition intended to be administered by injection can be prepared by combining lurbinectedin with water, or other physiologically suitable diluent, such as phosphate buffered saline, so as to form a solution. A surfactant can be added to facilitate the formation of a homogeneous solution or suspension.

Preferred compositions according to the present invention include:
Pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.
Lyophilised pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.

The ratio of lurbinectedin to the disaccharide in embodiments of the present invention is determined according to the solubility of the disaccharide and, when the formulation is freeze dried, also according to the freeze-dryability of the disaccharide. It is envisaged that this lurbinectedin:disaccharide ratio (w/w) can be about 1:10 in some embodiments, about 1:20 in other embodiments, about 1:50 in still other embodiments. It is envisaged that other embodiments have such ratios in the range from about 1:5 to about 1:500, and still further embodiments have such ratios in the range from about 1:10 to about 1:500.

The composition comprising lurbinectedin according to the present invention may be lyophilized. The composition comprising lurbinectedin is usually presented in a vial which contains a specified amount of such compound.

The following Example further illustrates the invention. It should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### EXAMPLES

In order to evaluate the efficacy of lurbinectedin in the treatment of progressive malignant pleural mesothelioma (MPM), a Phase II single-arm, multi-centric, international trial was performed.

### Methods

### Outcomes

The primary endpoint was progression-free survival at 12 weeks (PFS_{12wks}), defined as the absence of progression or death due to any cause during the first 12 weeks (±2 weeks) after registration. Secondary endpoints included progression-free survival (PFS), defined as the time from registration until radiologic progression or death due to any cause, whichever occurs first; overall survival (OS), defined as the time from registration until death due to any cause; objective response rate (ORR), defined as the proportion of patients who achieved complete (CR) or partial remission (PR) during the trial treatment; disease control rate (DCR) defined as the sum of CR, PR and stable disease (SD) at least for 12 weeks (SD≥_{12wks}); duration of disease control, defined as the time from registration to progression or death due to any cause whichever occurs first for patients who had CR, PR or SD_{≥12wks}; and adverse events (AE). All AEs were assessed according to NCI CTCAE v4.03. Patients lost to follow-up were censored at the last date they were known to be alive.

### Study Population

This trial was conducted in 6 centers in Switzerland and 3 in Italy. Subjects enrolled in this clinical trial were adult (age ≥ 18 years) patients having histologically or cytologically confirmed MPM, an Eastern Cooperative Oncology Group (ECOG) status of 0-1 and had progressed on or after one line of platinum-pemetrexed chemotherapy without being amenable to local treatment. Further inclusion criteria required an adequate hepatic, renal and bone marrow function (absolute neutrophil count ≥ 2 × 10⁹ /L, platelet count ≥ 100 × 10⁹ /L; aspartate aminotransferase and alanine aminotransferase ≤ 3.0 × ULN; creatinine clearance ≥ 30 mL/min/1.73).

Eligible patients could additionally have received one prior line of immunotherapy, but more than one lines of chemotherapy, including pemetrexed rechallenge, were not allowed. Further exclusion criteria covered known brain metastases or leptomeningeal disease, history of other hematologic or primary solid tumors within five years prior to registration (with the exception of curatively treated basal or squamous cell carcinoma of the skin, properly treated in situ malignant melanoma, in situ carcinoma of the uterine cervix or pT1-2 prostate cancer with Gleason score ≤6), concomitant use of anti-cancer surgery or radiotherapy (except for local pain control or pleurodesis) and adverse events Grade ≥2 derived from previous treatment.

### Procedures

Lurbinectedin was given every 3 weeks at a dose of 3.2 mg/m² as a 1-hour intravenous infusion through peripheral or central lines. Standard antiemetic prophylaxis (corticosteroids or serotonin (5-HT3) antagonists) was administered prior to chemotherapy. Due to potential significant interaction of lurbinectedin with aprepitant, as suggested by available Phase I and II clinical data in ovarian cancer trials, as reported by Poveda et al. (J. Clin Oncol 2014, 32:52 (suppl; abstr 5505), the use of aprepitant as anti-emetic prophylaxis was forbidden. Lurbinectedin was continued until progression, unacceptable toxicity or withdrawal of patient’s consent. In case of emergence of Grade ≥2 toxicity treatment was withheld until resolution to Grade 0-1; for Grade 3-4 toxicity there was additionally a dose reduction to 2.6 mg/m² every 3 weeks (first occurrence) or 2.0 mg/m² (second occurrence). In case of third re-emergence of Grade 3-4 toxicity treatment was permanently discontinued. Specifically for leucopenia Grade 3-4 and/or febrile neutropenia there was mandatory secondary G-CSF prophylaxis together with dose reduction by one level.

Patients were followed clinically and with laboratory tests at time of chemotherapy administration and additionally with contrast enhanced CT- or PET/CT scans every 6 weeks. Radiology scans were evaluated locally according to Response Evaluation Criteria in Solid Tumors (RECIST) modified for malignant mesothelioma, as showed by Byrne et al. (Ann Oncol, 2004, 15, 257-260). In case of treatment discontinuation for any reason, adverse events were reported for 30 days after last treatment application. For those cases where lurbinectedin was stopped without manifest progression radiology scans were performed every 8 weeks during the follow-up period otherwise patients were followed-up via telephone every 12 weeks for survival status.

### Statistical Analysis

A Simon two-stage design was used as presented by Simon R. (Control Clin Trials. 1989 Mar;10(1):1-10). We had set our null-hypothesis p₀ at PFS_{12wks} ≤35%, translated to a median PFS of ≤ 2 months. With a significance level of 5% and a power of 80%, the total sample size was calculated to be 42 patients taking into account 10% (4 patients) of non-evaluable patients.

An interim efficacy analysis, based on the first stage, was planned and Herndon’s approach (Control Clin Trials, 1998, 19, 5, 440-50) was applied to continue the accrual in the second stage. The trial would be stopped early if ≤7 patients experienced PFS_{12wks} or due to emergence of unacceptable toxicity regardless the efficacy result. At the end of the trial the treatment would be considered promising if p₀ was rejected, meaning the lower bound of 90% confidence interval (CI) of PFS_{12wks} been higher than 35%.

Baseline characteristics of the patients and treatment duration were descriptively summarized. PFS_{12wks} was estimated using the uniformly minimum variance unbiased estimator, as illustrated by Jung et al. (Stat Med. 2004 Mar 30; 23(6): 881-96), together with its corresponding 90% confidence interval (CI). Time-to-event endpoints were calculated using the Kaplan-Meier method with its corresponding 95% CI. Categorical endpoints were descriptively summarized with their corresponding 95% CI using the Clopper-Pearson method. Survival curves and rates between groups were compared using the log rank test and the Kaplan Meier method at a specific time point, respectively. Effect of subsequent treatment on the survival was evaluated using a non-proportional hazard model, as discussed by Fisher et al. (Annu Rev Public Health. 1999; 20:145-57). Statistical significance was set at p-value < 0.05. All analyses were performed using SAS ® 9.4 (SAS Institute Inc.) and R v3.5.1.

### Results

### Patients

A total of 42 patients were enrolled. Table 1 provides patient’s baseline characteristics together with information on prior treatment. Median age was 68.0 years (range 52-84) and the majority (83.3%) was male. Epithelioid histology was seen in 33 (78.6%) and non-epithelioid in 9 patients (21.4%) (4 biphasic (9.5%), 5 sarcomatoid (11.9%)). Regarding prior systemic treatment 32/42 patients (76.2%) had received platinum-pemetrexed alone whereas the rest 10/42 (23.8%) had received additionally one line of immunotherapy.

TABLE 1

| Characteristics | Overall (N=42) |
|---|---|
| Median Age (range), years | 68.0 (52.0, 84.0) |
| Sex | |
| Female | 7 (16.7%) |
| Male | 35 (83.3%) |
| ECOG performance status | |
| 0 | 20 (47.6%) |
| 1 | 22 (52.4%) |
| Histology at initial diagnosis | |
| Epithelioid | 33 (78.6%) |
| Biphasic | 4 (9.5%) |
| Sarcomatoid | 5 (11.9%) |
| Previous immunotherapy | |
| No | 32 (76.2%) |
| Yes | 10 (23.8%) |
| Time to progression on previous platinum-based chemotherapy | |
| ≥ 6 months | 28 (66.7%) |
| < 6 months | 14 (33.3%) |

### Outcome

After the analysis of the first 21 patients according to Simon’s 2-stage design, 11 patients (52.4%) had reached PFS_{12wks} so that study continued for full accrual. Towards end of the trial accrual was accelerated, so that ultimately a total of 42 were already included before stopping informing further patients.

Lurbinectedin was administered at a median of five cycles (range, 1-22) for 98 days (range, 22-525).

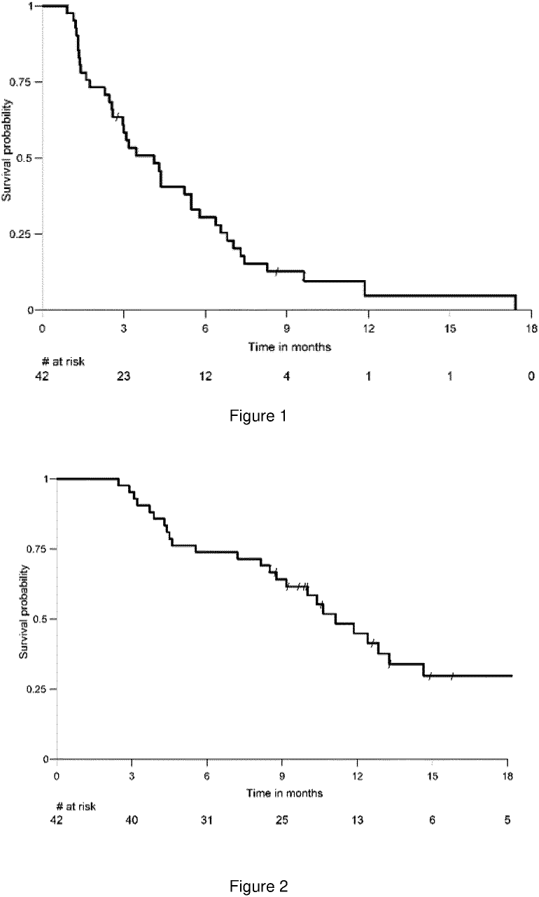

The primary endpoint of PFS_{12wks} was reached by 22/42 patients (52.4%; 90% CI: 38.7% - 63.5%, p=0.015). One patient showed CR, one PR and 20 patients had stable disease for an ORR of 4.8% and DCR of 52.4%. Using the Kaplan-Meier method at 12 weeks, PFS_{12wks} was 63.5% (90% CI: 49.7% - 74.4%). After a median follow-up time of 14.9 months, median PFS was 4.1 months (95% CI: 2.6 - 5.5) whereas 3, 6 and 9 months PFS% was 58.4% (95% CI: 41.8% - 71.7%), 30.5% (95% CI: 17.1% - 44.9%) and 12.7% (95% CI: 4.7% - 24.9%) respectively, as shown in FIG. 1. Median duration of disease control was 6.6 months (95% CI: 5.2 - 7.4) and 6/22 patients (27.3%) had a disease control lasting for 8 months or longer.

Upon progression on lurbinectedin 24 patients had received subsequent systemic treatment, as shown in Table 2.

TABLE 2

| | Overall (N=24) |
|---|---|
| Subsequent systemic treatment | n (%) |
| Alimta | 1 (4.2%) |
| Bevacizumab | 1 (4.2%) |
| Gemcitabine | 8 (33.3%) |
| Immune checkpoint inhibitor | 9 (37.5%) |
| Liposomal Doxorubicin | 1 (4.2%) |
| Platinum/pemetrexed | 8 (33.3%) |
| Vinorelbine | 7 (29.2%) |

At the time of analyses 26 patients had died (progression: 23; viral pneumonitis: 1; suicide: 1; heart failure: 1). Median OS was 11.1 months (95% CI: 8.8 - 14.7) and 6 and 12 months OS was 73.8% (95% CI: 57.7% - 84.6%) and 44.9% (95% CI: 28.1% -60.3%) respectively, as shown in FIG. 2. Patients who received subsequent systemic treatment did not live statistically significantly longer than the rest (p =0.74).

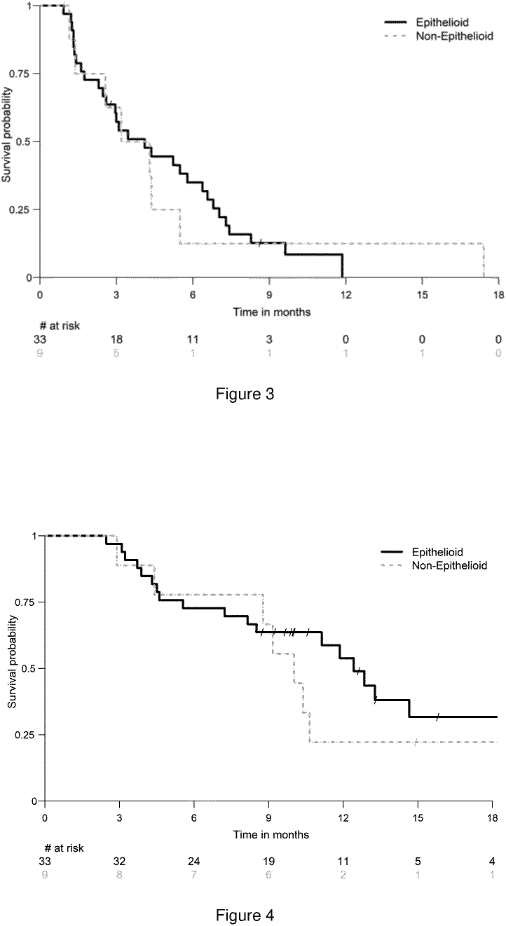

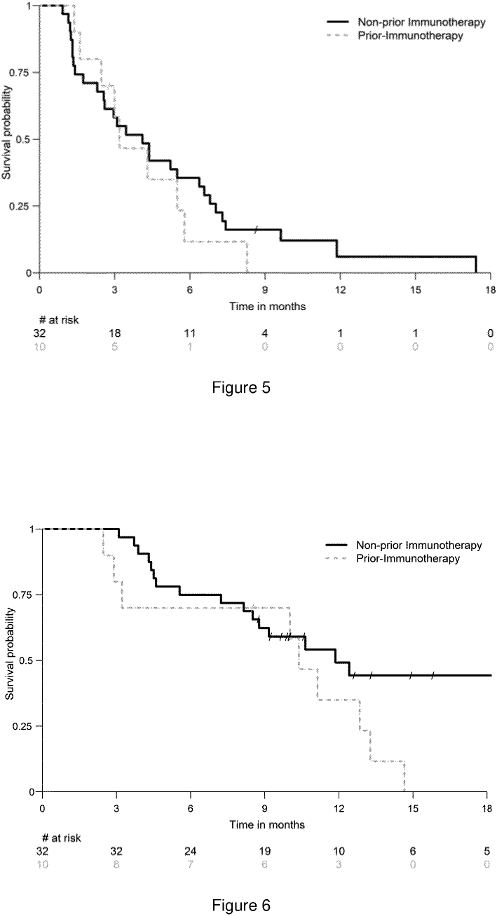

After evaluation of the impact of histology on treatment outcome no significant difference were observed in PFS_{12wks} (63.6% vs 62.5%, p=0.952), median PFS (4.1 vs 3.7 months, p=0.916) and median OS (12.4 vs 10.0 months, p=0.562) between epithelioid vs non-epithelioid histology respectively (FIGS. 3 and 4). Same was true for patients having received chemotherapy alone vs the ones with additional prior immunotherapy (PFS_{12wks}: 61.4% vs 70% (p=0.628), median PFS: 4.1 vs 3.2 months (p=0.508) and median OS 11.9 vs 10.4 months (p=0.099) respectively (FIGS. 5 and 6).

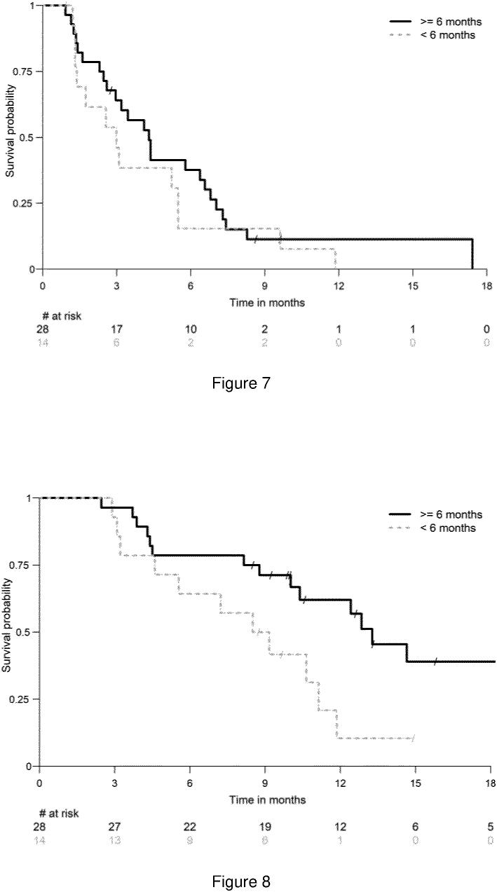

14 patients had progressed on previous platinum-pemetrexed chemotherapy within <6 months and 28 in ≥ 6 months. PFS_{12wks} and median PFS were similar (53.8% vs 67.9%, p=0.381; 3.0 vs 4.3 months, p=0.349, respectively), as shown in FIG. 7. Median OS was significantly longer in the group with a ≥ 6 months progression-free interval on prior pemetrexed (13.3 vs 8.8 months, p=0.023), as shown in FIG. 8.

### Toxicity

All patients (100%) experienced at least one adverse event (AE) of any grade and treatment related AEs (any grade) have been observed in 38/42 patients (90.5%), as shown in Table 3.

TABLE 3

Overall (N = 42)

| Grade | AEs n (%) | Lurbinectedin-related AEs n (%)¹ |
|---|---|---|
| 1 | 2 (48%) | 5 (11.9%) |
| 2 | 8(19.0%) | 13 (31.0%) |
| 3 | 24 (57.1%) | 0.13 (31.0%) |
| 4 | 8 (19.0%) | 7 (16.7%) |
| 5 | 0 (0.0%) | 0.0 (0.0%) |

AE: adverse event; ¹: 4 patients (9.5%) showed no lurbinectedin-related AEs

Lurbinectedin-related AEs Grade 3-4 toxicity were seen in in 20/42 patients (47.6%). There was no treatment-related death. The most common lurbinectedin-related AEs Grade 3-4 were neutropenia (23.8%) and fatigue (16.7%), but febrile neutropenia was documented in only 9.5% of the cases. All other Grade 3-4, lurbinectedin-related toxicities were < 10%, as shown in Table 4.

TABLE 4

| | | Overall (N = 42) | | | |
|---|---|---|---|---|---|
| | | Grade 1 | Grade 2 | Grade 3 | Grade 4 |
| Category | Term | n (%) | n (%) | n (%) | n (%) |
| Blood and lymphatic system disorders | Anemia | 3 (7.1%) | 4 (9.5%) | 3 (7.1%) | - |
| | Febrile neutropenia | | - | 4 (9.5%) | - |
| | Pancytopenia | - | 1 (2.4%) | - | - |
| Ear and labyrinth disorders | Vertigo | 1 (2.4%) | - | - | - |
| Eye disorders | Watering eyes | 1 (2.4%) | - | - | - |
| Gastrointestinal disorders | Abdominal pain | 1 (2.4%) | - | - | - |
| | Constipation | 9 (21.4%) | 1 (2.4%) | - | - |
| | Diarrhea | 3 (7.1%) | - | - | - |
| | Dyspepsia | 1 (2.4%) | - | - | - |
| | Dysphagia | 1 (2.4%) | 1 (2.4%) | - | - |
| | Halitosis | - | 1 (2.4%) | - | - |
| | Mucositis oral | 1 (2.4%) | - | - | - |
| | Nausea | 9 (21.4%) | 11 (26.2%) | 2 (4.8%) | - |
| | Vomiting | 9 (21.4%) | - | 2 (4.8%) | - |
| General disorders and administration site conditions | Edema limbs | 1 (2.4%) | - | - | - |
| | Fatigue | 7 (16.7%) | 11 (26.2%) | 7 (16.7%) | - |
| | Fever | 2 (4.8%) | 1 (2.4%) | - | - |
| | Flu like symptoms | 1 (2.4%) | - | - | - |
| Hepatobiliary disorders | Cholangitis | - | - | 1 (2.4%) | - |
| Infections and infestations | Infection of unknown origin | - | - | 1 (2.4%) | - |
| | Infections with unknown origine | - | 1 (2.4%) | - | - |
| | Lung infection | - | - | 1 (2.4%) | - |
| | Skin infection | - | 1 (2.4%) | - | - |
| | herpes zoster | - | 1 (2.4%) | - | - |
| Investigations | Alanine aminotransferase increased | 6 (14.3%) | 2 (4.8%) | - | - |
| | Alkaline phosphatase increased | 1 (2.4%) | - | - | - |
| | Aspartate aminotransferase increased | 2 (4.8%) | - | - | - |
| | Blood bilirubin increased | 3 (7.1%) | - | - | - |
| | Creatinine increased | 1 (2.4%) | 1 (2.4%) | - | - |
| | GGT increased | 4 (9.5%) | 2 (4.8%) | | - |
| | Neutrophil count decreased | - | 1 (2.4%) | 4 (9.5%) | 6 (14.3%) |
| | Platelet count decreased | 2 (4.8%) | 1 (2.4%) | 2 (4.8%) | 1 (2.4%) |
| | Urea increased | 1 (2.4%) | - | - | - |
| | Weight loss | 2 (4.8%) | - | - | - |
| | White blood cell decreased | - | 3 (7.1%) | 3 (7.1%) | - |
| Metabolism and nutrition disorders | Anorexia | 5 (11.9%) | 2 (4.8%) | 2 (4.8%) | - |
| | Hypomagnesemia | 5 (11.9%) | - | - | - |
| Musculoskeletal and connective tissue disorders | Generalized muscle weakness | 3 (7.1%) | 2 (4.8%) | 1 (2.4%) | - |
| Nervous system disorders | Dizziness | 2 (4.8%) | - | - | - |
| | Dysgeusia | - | 1 (2.4%) | - | - |
| | Headache | 1 (2.4%) | - | - | - |
| | Memory impairment | 1 (2.4%) | - | - | - |
| | Peripheral sensory neuropathy | 2 (4.8%) | 1 (2.4%) | - | - |
| Psychiatric disorders | Confusion | 1 (2.4%) | - | - | - |
| | Insomnia | - | 1 (2.4%) | - | - |
| Respiratory, thoracic and mediastinal disorders | Cough | 1 (2.4%) | - | - | - |
| | Dyspnea | 1 (2.4%) | 1 (2.4%) | 2 (4.8%) | - |
| | Productive cough | 1 (2.4%) | - | - | - |
| Skin and subcutaneous tissue disorders | Dry skin | 2 (4.8%) | - | - | - |
| Vascular disorders | Hypertension | 1 (2.4%) | 1 (2.4%) | - | - |
| | Superficial thrombophlebitis | - | 1 (2.4%) | - | - |

12/42 patients (28.6%) had at least one dose reduction during their treatment course and 2/42 patients experienced two dose reductions. There was no treatment discontinuation due to toxicity. Reason for dose reduction were AEs in 11/12 cases (91.7%) and physician’s decision in 1 case (8.3%). In the 10/11 patients where dose was reduced due to grade 3 — 4 AEs there was no re-emergence of grade 3 - 4 toxicity after dose-reduction and/or G-CSF application, whereas the 11^{th} patient had a second dose-reduction due to further grade 3 - 4 toxicity. Treatment was delayed at least once in 21/42 patients (50%). In descending order, causes for that were AEs (26.2%), patient’s decision (23.8%), administrative reasons (14.3%), physician’s decision (7.1%) and delay of necessary tumor assessment (4.8%).

From this study it can be concluded that the administration of lurbinectedin is safe and well tolerated with lurbinectedin showing an acceptable safety profile. The trial showed promising clinical activity of lurbinectedin in malignant mesothelioma with acceptable toxicity. Lurbinectedin is shown to work independent of histology or prior immunotherapy. In addition, both “slow” and “fast” progressive patients from platinum-pemetrexed prior therapy are shown to benefit from lurbinectedin.

The data demonstrates that lurbinectedin as a monotherapy is safe and efficacious in the treatment of malignant mesothelioma. The data demonstrates that lurbinectedin monotherapy reversed, attenuated, or inhibited disease progression in a challenging patient group who have progressed from standard therapy and who would otherwise have a very poor prognosis. This surprising finding confirms the usefulness of lurbinectedin as a monotherapy in the treatment of malignant mesothelioma per se, including for example both malignant pleural mesothelioma and malignant peritoneal mesothelioma. Particularly, the data demonstrates the effectiveness of lurbinectedin monotherapy in the treatment of progressive malignant mesothelioma, malignant pleural mesothelioma, and progressive malignant pleural mesothelioma. The data demonstrates that lurbinectedin is effective when not administered as a combination therapy with a platinum agent, for example cisplatin. Thus, the data confirms the usefulness of lurbinectedin administered in the absence of a platinum agent such as cisplatin in the treatment of malignant mesothelioma per se, including for example both malignant pleural mesothelioma and malignant peritoneal mesothelioma.

While epithelioid mesotheliomas normally exhibit a better outcome than mixed or sarcomatoid mesotheliomas, the data shows that there was no difference in the outcome between epithelioid and non-epithelioid malignant pleural mesothelioma. This suggests that lurbinectedin is likely to equalise the prognosis of these subtypes of malignant mesothelioma.

The data also shows that G-CSF diminished the incidence of grade 3-4 neutropenia.

1-29. (canceled)

30. A method of treating malignant mesothelioma in a patient in need thereof, comprising administering to said patient an effective amount of lurbinectedin or a pharmaceutically acceptable salt or ester thereof as a monotherapy.

31. A method of treating malignant mesothelioma in a patient in need thereof, comprising administering to said patient an effective amount of lurbinectedin or a pharmaceutically acceptable salt or ester thereof, wherein lurbinectedin treatment excludes treatment with a combination of lurbinectedin and a platinum agent.

32. The method of claim 31, wherein the platinum agent is cisplatin.

33. A method of treating malignant mesothelioma in a patient in need thereof, comprising administering to said patient an effective amount of lurbinectedin or a pharmaceutically acceptable salt or ester thereof, wherein lurbinectedin or a pharmaceutically acceptable salt is the sole chemotherapy agent.

34. The method according to claim 30, wherein the malignant mesothelioma is progressive, preferably wherein the malignant mesothelioma has progressed from first-line therapy, further preferably wherein the malignant mesothelioma has progressed from standard first-line therapy.

35. The method according to claim 34, wherein the first-line therapy is platinum-pemetrexed chemotherapy; optionally including surgery and/or radiotherapy.

36. The method according to claim 34, wherein the first-line therapy is an immunotherapy, preferably an anti-PD-1, anti-PD-L1 or anti CTLA-4 therapy, or combinations thereof.

37. The method according to claim 34, wherein the first-line therapy is platinum-pemetrexed chemotherapy and an immunotherapy; wherein the immunotherapy is preferably an anti-PD-1, anti-PD-L1 or anti CTLA-4 therapy, of combinations thereof.

38. The method according to claim 34, wherein lurbinectedin is administered following immunotherapy; preferably an anti-PD-1, anti-PD-L1 or anti CTLA-4 therapy, or combinations thereof.

39. The method according to claim 34, wherein the first-line therapy is an anti-angiogenesis therapy; preferably wherein the anti-angiogenesis therapy is a VEGF inhibitor, such as bevacizumab.

40. The method according to claim 30, wherein the treatment is a third-line therapy; optionally wherein the previous lines of therapy are selected from:
platinum-pemetrexed chemotherapy; optionally including surgery and/or radiotherapy;
immunotherapy, preferably an anti-PD-1, anti-PD-L1 or anti CTLA-4 therapy, or combinations thereof; and
an anti-angiogenesis therapy; preferably wherein the anti-angiogenesis therapy is a VEGF inhibitor, such as bevacizumab.

41. The method of claim 40, wherein the first line therapy is platinum-pemetrexed chemotherapy and the second line therapy is immunotherapy.

42. The method according to claim 30, wherein lurbinectedin is administered once every one to four weeks, preferably once every three weeks; and/or
wherein lurbinectedin is administered at a dose of 1 to 5 mg/m2 body surface area, 2 to 3 mg/m2 body surface area, about 3 mg/m2 body surface area, 3 to 3.5 mg/m2 body surface area, or 3.2 mg/m2 body surface area; and/or
wherein lurbinectedin is administered as an infusion, preferably with an infusion time of up to 24 hours, 1 to 12 hours, 1 to 6 hours and most preferably 1 hour.

43. The method according to claim 30, wherein the patient is additionally treated with radiotherapy; optionally wherein the radiotherapy is administered prior to or subsequent to administration of lurbinectedin or a pharmaceutically acceptable salt or ester thereof, preferably at least an hour, three hours, five hours, 12 hours, a day, a week, a month, more preferably several months (e.g. up to three months) prior or subsequent to administration of lurbinectedin or a pharmaceutically acceptable salt or ester thereof.

44. The method according to claim 30, wherein the patient is additionally treated with an anti-emetic, G-CSF and/or GM-CSF, preferably wherein the patient is additionally treated with G-CSF.

45. The method according to claim 30, wherein the malignant mesothelioma is selected from the list consisting of malignant pleural mesothelioma, malignant peritoneal mesothelioma, epithelioid mesothelioma, sarcomatoid mesothelioma and biphasic mesothelioma.

46. The method according to claim 30, wherein the pharmaceutically acceptable salt is selected from the hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate p-toluenesulfonate, sodium, potassium, calcium and ammonium salts, ethylenediamine, ethanolamine, N,N-dialkylenethanolamine, triethanolamine and basic aminoacids salts.

47. The method according to claim 31, wherein the malignant mesothelioma is progressive, preferably wherein the malignant mesothelioma has progressed from first-line therapy, further preferably wherein the malignant mesothelioma has progressed from standard first-line therapy.

48. The method according to claim 47, wherein the first-line therapy is platinum-pemetrexed chemotherapy; optionally including surgery and/or radiotherapy.

49. The method according to claim 47, wherein the first-line therapy is an immunotherapy, preferably an anti-PD-1, anti-PD-L1 or anti CTLA-4 therapy, or combinations thereof.

50. The method according to claim 47, wherein the first-line therapy is platinum-pemetrexed chemotherapy and an immunotherapy; wherein the immunotherapy is preferably an anti-PD-1, anti-PD-L1 or anti CTLA-4 therapy, of combinations thereof.

51. The method according to claim 47, wherein lurbinectedin is administered following immunotherapy; preferably an anti-PD-1, anti-PD-L1 or anti CTLA-4 therapy, or combinations thereof.

52. The method according to claim 47, wherein the first-line therapy is an anti-angiogenesis therapy; preferably wherein the anti-angiogenesis therapy is a VEGF inhibitor, such as bevacizumab.

53. The method according to claim 31, wherein the treatment is a third-line therapy; optionally wherein the previous lines of therapy are selected from:
platinum-pemetrexed chemotherapy; optionally including surgery and/or radiotherapy;
immunotherapy, preferably an anti-PD-1, anti-PD-L1 or anti CTLA-4 therapy, or combinations thereof; and
an anti-angiogenesis therapy; preferably wherein the anti-angiogenesis therapy is a VEGF inhibitor, such as bevacizumab.

54. The method of claim 53, wherein the first line therapy is platinum-pemetrexed chemotherapy and the second line therapy is immunotherapy.

55. The method according to claim 31, wherein lurbinectedin is administered once every one to four weeks, preferably once every three weeks; and/or
wherein lurbinectedin is administered at a dose of 1 to 5 mg/m2 body surface area, 2 to 3 mg/m2 body surface area, about 3 mg/m2 body surface area, 3 to 3.5 mg/m2 body surface area, or 3.2 mg/m2 body surface area; and/or
wherein lurbinectedin is administered as an infusion, preferably with an infusion time of up to 24 hours, 1 to 12 hours, 1 to 6 hours and most preferably 1 hour.

56. The method according to claim 31, wherein the patient is additionally treated with radiotherapy; optionally wherein the radiotherapy is administered prior to or subsequent to administration of lurbinectedin or a pharmaceutically acceptable salt or ester thereof, preferably at least an hour, three hours, five hours, 12 hours, a day, a week, a month, more preferably several months (e.g. up to three months) prior or subsequent to administration of lurbinectedin or a pharmaceutically acceptable salt or ester thereof.

57. The method according to claim 31, wherein the patient is additionally treated with an anti-emetic, G-CSF and/or GM-CSF, preferably wherein the patient is additionally treated with G-CSF.

58. The method according to claim 31, wherein the malignant mesothelioma is selected from the list consisting of malignant pleural mesothelioma, malignant peritoneal mesothelioma, epithelioid mesothelioma, sarcomatoid mesothelioma and biphasic mesothelioma.

59. The method according to claim 31, wherein the pharmaceutically acceptable salt is selected from the hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate p-toluenesulfonate, sodium, potassium, calcium and ammonium salts, ethylenediamine, ethanolamine, N,N-dialkylenethanolamine, triethanolamine and basic aminoacids salts.

60. The method according to claim 33, wherein the malignant mesothelioma is progressive, preferably wherein the malignant mesothelioma has progressed from first-line therapy, further preferably wherein the malignant mesothelioma has progressed from standard first-line therapy.

61. The method according to claim 60, wherein the first-line therapy is platinum-pemetrexed chemotherapy; optionally including surgery and/or radiotherapy.

62. The method according to claim 60, wherein the first-line therapy is an immunotherapy, preferably an anti-PD-1, anti-PD-L1 or anti CTLA-4 therapy, or combinations thereof.

63. The method according to claim 60, wherein the first-line therapy is platinum-pemetrexed chemotherapy and an immunotherapy; wherein the immunotherapy is preferably an anti-PD-1, anti-PD-L1 or anti CTLA-4 therapy, of combinations thereof.

64. The method according to claim 60, wherein lurbinectedin is administered following immunotherapy; preferably an anti-PD-1, anti-PD-L1 or anti CTLA-4 therapy, or combinations thereof.

65. The method according to claim 60, wherein the first-line therapy is an anti-angiogenesis therapy; preferably wherein the anti-angiogenesis therapy is a VEGF inhibitor, such as bevacizumab.

66. The method according to claim 33, wherein the treatment is a third-line therapy; optionally wherein the previous lines of therapy are selected from:
platinum-pemetrexed chemotherapy; optionally including surgery and/or radiotherapy;
immunotherapy, preferably an anti-PD-1, anti-PD-L1 or anti CTLA-4 therapy, or combinations thereof; and
an anti-angiogenesis therapy; preferably wherein the anti-angiogenesis therapy is a VEGF inhibitor, such as bevacizumab.

67. The method of claim 66, wherein the first line therapy is platinum-pemetrexed chemotherapy and the second line therapy is immunotherapy.

68. The method according to claim 33, wherein lurbinectedin is administered once every one to four weeks, preferably once every three weeks; and/or
wherein lurbinectedin is administered at a dose of 1 to 5 mg/m2 body surface area, 2 to 3 mg/m2 body surface area, about 3 mg/m2 body surface area, 3 to 3.5 mg/m2 body surface area, or 3.2 mg/m2 body surface area; and/or
wherein lurbinectedin is administered as an infusion, preferably with an infusion time of up to 24 hours, 1 to 12 hours, 1 to 6 hours and most preferably 1 hour.

69. The method according to claim 33, wherein the patient is additionally treated with radiotherapy; optionally wherein the radiotherapy is administered prior to or subsequent to administration of lurbinectedin or a pharmaceutically acceptable salt or ester thereof, preferably at least an hour, three hours, five hours, 12 hours, a day, a week, a month, more preferably several months (e.g. up to three months) prior or subsequent to administration of lurbinectedin or a pharmaceutically acceptable salt or ester thereof.

70. The method according to claim 33, wherein the patient is additionally treated with an anti-emetic, G-CSF and/or GM-CSF, preferably wherein the patient is additionally treated with G-CSF.

71. The method according to claim 33, wherein the malignant mesothelioma is selected from the list consisting of malignant pleural mesothelioma, malignant peritoneal mesothelioma, epithelioid mesothelioma, sarcomatoid mesothelioma and biphasic mesothelioma.

72. The method according to claim 33, wherein the pharmaceutically acceptable salt is selected from the hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate p-toluenesulfonate, sodium, potassium, calcium and ammonium salts, ethylenediamine, ethanolamine, N,N-dialkylenethanolamine, triethanolamine and basic aminoacids salts.

73. A kit comprising lurbinectedin or a pharmaceutically acceptable salt or ester thereof together with instructions for treating malignant mesothelioma, wherein the instructions are according to claim 30.

74. A kit comprising lurbinectedin or a pharmaceutically acceptable salt or ester thereof together with instructions for treating malignant mesothelioma, wherein the instructions are according to claim 31.

75. A kit comprising lurbinectedin or a pharmaceutically acceptable salt or ester thereof together with instructions for treating malignant mesothelioma, wherein the instructions are according to claim 33.

76. A method of inhibiting cancer cell growth, wherein the cancer is malignant mesothelioma, comprising contacting cancer cells with lurbinectedin or a pharmaceutically acceptable salt or ester thereof:
wherein lurbinectedin or a pharmaceutically acceptable salt or ester thereof is contacted to the cancer cells as a monotherapy; or
wherein the cancer cells are not and have not been contacted with a platinum agent; optionally wherein the platinum agent is cisplatin; or
wherein lurbinectedin or a pharmaceutically acceptable salt is the sole chemotherapy agent applied to the cancer cells.
